(19) United States
(12) Patent Application Publication
(10) Pub. No.: US 2025/0375538 A1
(43) Pub. Date: Dec. 11, 2025

(54) COMPOSITIONS AND METHODS FOR IMPROVED mRNA PRODUCTION

(71) Applicant: The Hong Kong University of Science and Technology, Kowloon (CN)

(72) Inventors: Yi KUANG, Kowloon (CN); Cheuk Yin LI, Kowloon (CN); Lala FU, Tseung Kwan O (CN)

(21) Appl. No.: 19/227,388

(22) Filed: Jun. 3, 2025

Related U.S. Application Data
(60) Provisional application No. 63/656,577, filed on Jun. 5, 2024.

(51) Int. Cl. A61K 48/00; C12N 15/85

(52) U.S. Cl. CPC A61K 48/0066; C12N 15/85; C12N 2830/50

(57) ABSTRACT

The present invention discloses a modified poly(A) sequence for use in a recombinantly produced mRNA molecule for the purpose of improving the production process of the mRNA and subsequent protein expression from the mRNA. Thus, new and effective compositions and methods are provided for use in various applications involving improved production process of an mRNA of interest as well as enhanced expression of the mRNA-encoded protein.

### RELATED APPLICATIONS

This application claims priority to U.S. Provisional Patent Application No. 63/656,577, filed Jun. 5, 2024, the contents of which are hereby incorporated by reference in the entirety for all purposes.

### SEQUENCE LISTING

A Sequence Listing conforming to the rules of WIPO Standard ST.26 is hereby incorporated by reference. Said Sequence Listing has been filed as an electronic document via PatentCenter encoded as XML in UTF-8 text. The electronic document, created on Aug. 15, 2025, is entitled “091256-1493582-004310US_ST26.xml”, and is 7,937 bytes in size.

### BACKGROUND OF THE INVENTION

Messenger RNA (mRNA) is a key molecule in the flow of genetic information. mRNAs are long nucleotide chains that encode protein information from the genome. They produce all the proteins in the cell and are therefore one of the essential biomolecules of life. While mRNAs have been the subject of basic biological research for half a century, only in the past two decades has it been recognized and developed to be a potentially new powerful therapeutic tool. Synthetic mRNA therapeutics have some advantages over DNA- and protein-based counterparts and are beginning to be used more frequently in recent years with commercial success. As mRNAs naturally degrade in the biological system, high dose or repeated administration is commonly required. Previous studies showed that use of artificial sequences or chemically modified nucleotides in mRNAs can increase mRNA stability and availability, thus enhancing mRNA therapeutics' performance. In particular, the present inventors have earlier demonstrated the successful use of modified poly(A) tail sequences for the purpose of improving recombinant protein expression, see, e.g., WO2022/028559, WO2024/188312, and WO2025/011636.

Considering the increased interest and usage of mRNA therapeutics, there remains a pressing need for new compositions and methods that can further improve the production process of mRNAs and ultimately increase the efficiency of recombinant protein expression from the mRNAs. This invention fulfills this and other related needs.

### BRIEF SUMMARY OF THE INVENTION

The use of a modified poly(A) tail in the form of a cytidine-containing tail sequence for the purpose of improving the production of a synthetic mRNA, for example, from a plasmid was previously reported (see, e.g., WO2022/028559, WO2024/188312, and WO2025/011636). This disclosure reports newly optimized cytosine-containing tail sequences and demonstrates that they are able to (1) minimize copy error during bacterial cloning of the plasmid; and (2) prolong and enhance the expression level of mRNA for mRNA-based therapeutics, including mRNA vaccines. Thus, the first aspect of this invention relates to an artificial poly(A) sequence that includes, from its 5′ end to its 3′ end, a first segment of about 20-60 adenines, a second segment or a linker sequence of about 5-20 nucleotides of any of adenine (A), cytosine (C), guanine (G), and thymine (T)/uracil (U), i.e., randomly selected nucleotides, a third segment of about 30-90 adenines, a fourth segment of about 5-40 cytosines, and lastly 1-5 adenines at its 3′ end. In some embodiments, the number of cytosines in this artificial poly(A) sequence is no more than ⅓ of the total number of nucleotides in this artificial poly(A) sequence, for example, the number of cytosines in this artificial poly(A) sequence is no more than 30% of the total number of this artificial poly(A) sequence. In some cases, the length of the 4th segment is no more than ⅓ of the total length of this artificial poly(A) sequence. In some embodiments, the artificial poly(A) sequence has about 25-50 adenines at its 5′ end, a linker of about 7-15 random nucleotides, about 40-80 adenines, about 7-20 cytosines, and about 1-3 adenines at its 3′ end. In some embodiments, the artificial poly(A) sequence has about 30 adenines at its 5′ end, a linker of about 10 random nucleotides, about 60 adenines, about 10 cytosines, and 1 adenine at its 3′ end, for example, it may have 30 adenines at its 5′ end, 1 adenine at its 3′ end, with 10 random nucleotides (e.g., SEQ ID NO:5), 59 adenines, and 10 cytosines in between. In some embodiments, the artificial poly(A) sequence consists of the nucleotide sequence set forth in SEQ ID NO:4. The artificial poly(A) sequence of this invention, described above and herein, may be a DNA sequence or an RNA sequence.

In a second aspect, the present invention provides nucleic acid constructs, which may be in the form of DNA or RNA, that supports mRNA transcription and/or protein expression from a coding sequence containing the artificial poly(A) sequence described above and herein. In some embodiments, an expression cassette is provided, which comprises a promoter and a polynucleotide sequence encoding the artificial poly(A) sequence described above and herein. In some embodiments, the expression cassette further comprises a multiple cloning site between the promoter and the polynucleotide sequence encoding the artificial poly(A) sequence. In some embodiments, the expression cassette further comprises a transcription initiation codon and a transcription termination codon, both operably linked to the promoter and the polynucleotide sequence encoding the artificial poly(A) sequence. In some embodiments, the expression cassette further comprises a polynucleotide sequence encoding one or more polypeptides between the promoter and the artificial poly(A) sequence, with the polynucleotide sequence operably linked to the promoter and the polynucleotide sequence encoding the artificial poly(A) sequence. In some embodiments, the artificial poly(A) sequence in the nucleic acid constructs of this invention (e.g., an expression cassette) consists of the nucleotide sequence set forth in SEQ ID NO:4.

In a related aspect, the present invention provides a vector, e.g., an expression vector, that comprises the expression cassette described above and herein. Such vectors or expression cassettes in some cases are DNA constructs. Also provided is a recombinant host cell that harbors the expression cassette or the vector of this invention as described above and herein, as well as a composition that comprises the expression cassette or the vector of this invention as described above and herein. In some embodiments, the artificial poly(A) tail sequence in the vector consists of the nucleotide sequence set forth in SEQ ID NO:4.

In a third aspect, the present invention provides methods for RNA transcription or recombinant protein production in a cell or a lysate of cells. For example, a method for RNA transcription includes these steps: (i) transfecting the cell with, or introducing into the cell lysate, the expression cassette or the vector of the present invention, as described above or herein; and (ii) cultivating the cell or maintaining the lysate under conditions permissible for RNA transcription from the expression cassette or the vector. In some embodiments, the method further includes a step of isolating the RNA transcribed in step (ii). In some embodiments, the cell is a bacterial cell or the cell lysate is a bacterial cell lysate, e.g., E. coli cell or E. coli cell lysate. In some embodiments, the cell is a mammalian cell or the cell lysate is a mammalian cell lysate, e.g., HEK293 cell or Hela cell or their lysate. In the case of a method for recombinant protein expression in a cell, typically included are step (i) transfecting the cell with the expression cassette, or the vector, or the RNA of the present invention, as described above or herein; and step (ii) cultivating or maintaining the cell under conditions permissible for protein expression from the expression cassette or the vector or the RNA of the present invention. In either method, an exemplary expression cassette, the vector, or the RNA may comprise a polynucleotide sequence encoding one or more proteins of interest. For example, the expression cassette, the vector, or the RNA may comprise an artificial poly(A) sequence having the nucleotide sequence of SEQ ID NO:4.

Depending on the specific application, any of these two methods may be practiced in vitro within intact cells (prokaryotic or eukaryotic cells) or in functional cell lysates or in vivo, for example, in mammalian cells, including human cells present within a human body.

In a further related aspect, the present invention provides an RNA molecule comprising a coding sequence for one or more polypeptides and the artificial poly(A) sequence as described above or herein. Also provided is an RNA molecule that is transcribed from the expression cassette or the vector of the present invention, as described above and herein. The artificial poly(A) sequence includes, from its 5′ end to its 3′ end, a first segment of about 20-60 adenines, a second segment or a linker sequence of about 5-20 nucleotides of any of A, C, G, or T/U, i.e., randomly selected nucleotides, a third segment of about 30-90 adenines, a fourth segment of about 5-40 cytosines, and lastly 1-5 adenines at its 3′ end. In some embodiments, the number of cytosines in this artificial poly(A) sequence is no more than ⅓ of the total number of nucleotides in this artificial poly(A) sequence, for example, the number of cytosines in this artificial poly(A) sequence is no more than 30% of the total number of this artificial poly(A) sequence. In some embodiments, the artificial poly(A) sequence has about 25-50 adenines at its 5′ end, a linker of about 7-15 random nucleotides, about 40-80 adenines, about 7-20 cytosines, and about 1-3 adenines at its 3′ end. In some embodiments, the artificial poly(A) sequence has about 30 adenines at its 5′ end, a linker of about 10 random nucleotides, about 60 adenines, about 10 cytosines, and 1 adenine at its 3′ end, for example, it may have 30 adenines at its 5′ end, 1 adenine at its 3′ end, with 10 random nucleotides (e.g., SEQ ID NO:5), 59 adenines, and 10 cytosines in between. In some embodiments, the artificial poly(A) sequence consists of the nucleotide sequence set forth in SEQ ID NO:4. In some embodiments, the RNA includes a coding sequence for one or more polypeptides of interest. For example, the encoded protein(s) of interest may serve a therapeutic or prophylactic purpose (e.g., a therapeutic protein useful for treating a disease or a pathogen-derived protein antigen as a vaccine to prevent future infection). In such cases, compositions comprising the RNA molecule of this invention as described above and herein are formulated in accordance with their intended uses, e.g., for injection or for local delivery such as via mucosal delivery through nasal or oral routes, to include at least one potentially more physiologically or pharmaceutically acceptable excipients or carriers. Moreover, in the case of any compositions intended for eliciting a desired immune response, one or more adjuvants known for their safe and effective use in the manufacturing of vaccines may be further included.

### BRIEF DESCRIPTION OF THE DRAWINGS

FIG. 1. Percentage of recombinant clone after bacteria amplification (n=20).

FIG. 2. OD600 of E. coli culture at 24 hours post-transformation (n=5); data are presented as mean±SD.

FIG. 3. Relative EGFP expression of HEK293 cells at 24 hours post-transfection (n=3): data are presented as mean±SD: the levels of significance are denoted as * p<0.05, **** p<0.0001.

### DEFINITIONS

As used herein, the term “artificial poly(A) sequence” refers to a polynucleotide containing a string of consecutive adenines (A), among which at least one is substituted with a non-adenine nucleotide, such as cytosine (C), guanine (G), and thymine (T)/uracil (U). Typically, the substitution involves multiple non-A nucleobases in one or two or more stretches of about 5 to about 30 nucleobases each in length, located within the last ¾ to ⅓ section of the entire sequence from its 3′ end, although the last nucleotide in the artificial poly(A) sequence is often not substituted and remains A.

The term “nucleic acid” or “polynucleotide” refers to deoxyribonucleotides or ribonucleotides and polymers thereof in either single- or double-stranded form. Unless specifically limited, the term encompasses nucleic acids containing known analogues of natural nucleotides which have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res., 19:5081 (1991); Ohtsuka et al., J. Biol. Chem., 260:2605-2608 (1985); and Cassol et al., (1992): Rossolini et al., Mol. Cell. Probes, 8:91-98 (1994)). The terms nucleic acid and polynucleotide are used interchangeably with gene, cDNA, and mRNA encoded by a gene.

The terms “polypeptide,” “peptide,” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymers. As used herein, the terms encompass amino acid chains of any length, including full length proteins or fragments thereof, wherein the amino acid residues are linked by covalent peptide bonds.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. “Amino acid mimetics” refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The term “expression cassette” refers to a nucleic acid construct, generated recombinantly or synthetically, with a series of specified nucleic acid elements that permit transcription of a particular polynucleotide sequence in a host cell. An expression cassette may be a part of a circular construct such as a plasmid, a viral genome or vector, or a longer nucleic acid fragment. Typically, an expression cassette includes a polynucleotide sequence to be transcribed, operably linked to a promoter (e.g., a heterologous promoter). “Operably linked” in this context means that two or more genetic elements, such as a polynucleotide coding sequence and a promoter, are placed in relative positions that permit the proper biological functioning of the elements, such as the promoter directing transcription of the coding sequence. Other elements (e.g., heterologous elements) that may be present in an expression cassette include those that enhance transcription (e.g., enhancers) and terminate transcription (e.g., terminators), as well as those that confer certain binding affinity or antigenicity to the recombinant protein produced from the expression cassette.

The term “heterologous,” as used in the context of describing the relative location of two elements, refers to the two elements such as two polynucleotide sequences (e.g., a promoter and a polypeptide-encoding sequence) or polypeptide sequences (e.g., a first amino acid sequence and a second peptide sequence serving as a fusion partner with the first amino acid sequence) that are not naturally found in the same relative position. Thus, the description of a “heterologous promoter” of a gene or coding sequence refers to a promoter that is not naturally found to be operably linked to that gene.

The term “multiple cloning site” refers to a short stretch of nucleotide sequence (e.g., about 20-50 nucleotides) comprising multiple restriction endonuclease recognition sites permitting enzymatic digestion and subsequent insertion of another sequence encoding an RNA or protein.

The term “inhibiting” or “inhibition,” as used herein, refers to any detectable negative effect on a target biological process, such as RNA/protein expression of a target gene, the biological activity of a target protein, cellular signal transduction, cell proliferation, presence/level of an organism especially a micro-organism, any measurable biomarker, bio-parameter, or symptom in a subject, and the like. Typically, an inhibition is reflected in a decrease of at least 10%, 20%, 30%, 40%, 50%, 60%, 70%, 80%, 90% or greater in the target process (e.g., a biomarker level, RNA transcription level, or protein expression level), or any one of the downstream parameters mentioned above, when compared to a control. “Inhibition” further includes a 100% reduction, i.e., a complete elimination, prevention, or abolition of a target biological process or signal. The other relative terms such as “suppressing,” “suppression,” “reducing,” and “reduction” are used in a similar fashion in this disclosure to refer to decreases to different levels (e.g., at least 10%, 20%, 30%, 40%, 50%, 60%, 70%, 80%, 90% or greater decrease compared to a control level) up to complete elimination of a target biological process or signal. On the other hand, terms such as “activate,” “activating,” “activation,” “increase,” “increasing,” “promote,” “promoting,” “enhance,” “enhancing,” or “enhancement” are used in this disclosure to encompass positive changes at different levels (e.g., at least about 5%, 10%, 20%, 30%, 40%, 50%, 60%, 70%, 80%, 90%, 100%, 200%, or greater such as 3, 5, 8, 10, 20-fold increase compared to a control level in a target process, signal, or parameter.

As used herein, the term “treatment” or “treating” includes both therapeutic and preventative measures taken to address the presence of a disease or condition or the risk of developing such disease or condition at a later time. It encompasses therapeutic or preventive measures for alleviating ongoing symptoms, inhibiting or slowing disease progression, delaying onset of symptoms, or eliminating or reducing side-effects caused by such disease or condition. A preventive measure in this context and its variations do not require 100% elimination of the occurrence of an event: rather, they refer to a suppression or reduction in the likelihood or severity of such occurrence or a delay in such occurrence.

The term “about” when used in reference to a given value denotes a range encompassing±10% of the value.

A “pharmaceutically acceptable” or “pharmacologically acceptable” excipient is a substance that is not biologically harmful or otherwise undesirable, i.e., the excipient may be administered to an individual along with a bioactive agent without causing any undesirable biological effects. Neither would the excipient interact in a deleterious manner with any of the components of the composition in which it is contained.

The term “excipient” refers to any essentially accessory substance that may be present in the finished dosage form of the composition of this invention. For example, the term “excipient” includes vehicles, binders, disintegrants, fillers (diluents), lubricants, adjuvants, glidants (flow enhancers), compression aids, colors, sweeteners, preservatives, suspending/dispersing agents, film formers/coatings, flavors and printing inks.

### DETAILED DESCRIPTION OF THE INVENTION

### I. Introduction

It was previously discovered that artificial poly(A) sequences with some adenines replaced with cytosines, when joined to the 3′ end of an RNA sequence, can effectively enhance protein expression from the RNA sequence. These artificial poly(A) sequences can improve RNA stability and therefore can enhance the performance of both simple and smart model mRNA drugs. See, e.g., WO2022/028559, WO2024/188312, and WO2025/011636. As the artificial poly(A) sequences can be simply incorporated into the DNA templates by regular PCR reactions, no additional cost is needed for synthesizing mRNA drugs carrying the artificial poly(A) sequences. The artificial poly(A) sequence can be used with other mRNA technologies including modified nucleotides, modified cap analog. Therefore, these artificial poly(A) sequences can be broadly used on the existing and future mRNA drugs for enhancement of efficacy and for reduction of cost.

The present inventors have now further improved the artificial poly(A) sequences containing A to C substitutions. The feature of C substitutions is described as follows: First, with a total nucleotide number of the artificial poly(A) sequence being n, the number of Cs in the artificial poly(A) sequence m is defined as 0.3n≥m≥1, with or without a linker consisting of a string of random nucleotides (e.g., about 5 to about 30 nucleotides, each position is randomly and independently selected from A, C, G, and T/U) located before the stretch of cytidines (i.e., to the 5′ end of the C stretch). For example, artificial poly(A) tail sequence without any linker is first described in WO2022/028559. Second, the C residues are located within the last 30% of the artificial poly(A) tail sequence from its 3′ end, excluding the last nucleotide location at the 3′ end. The C locations can be either adjacent to each other (forming a stretch, e.g., about 10 to about 30 in length) or separated from each other (e.g., with one or more adenines in between). The newly improved artificial poly(A) tail sequences disclosed herein are able to support both (1) a significantly higher fidelity in the replication of a DNA sequence encoding for an mRNA (e.g., contained in an expression vector such as a plasmid) by way of minimizing recombination rate thus copying error rate during DNA replication and (2) an enhanced protein expression level of the protein encoded by the mRNA.

### II. General Recombinant Technology

Basic texts disclosing general methods and techniques in the field of recombinant genetics include Sambrook and Russell, Molecular Cloning. A Laboratory Manual (3rd ed. 2001): Kriegler, Gene Transfer and Expression: A Laboratory Manual (1990); and Ausubel et al., eds., Current Protocols in Molecular Biology (1994).

For nucleic acids, sizes are given in either kilobases (kb) or base pairs (bp). These are estimates derived from agarose or acrylamide gel electrophoresis, from sequenced nucleic acids, or from published DNA sequences. For proteins, sizes are given in kilodaltons (kDa) or amino acid residue numbers. Proteins sizes are estimated from gel electrophoresis, from sequenced proteins, from derived amino acid sequences, or from published protein sequences.

Oligonucleotides that are not commercially available can be chemically synthesized, e.g., according to the solid phase phosphoramidite triester method first described by Beaucage & Caruthers, Tetrahedron Lett. 22:1859-1862 (1981), using an automated synthesizer, as described in Van Devanter et. al., Nucleic Acids Res. 12:6159-6168 (1984). Purification of oligonucleotides is performed using any art-recognized strategy, e.g., native acrylamide gel electrophoresis or anion-exchange HPLC as described in Pearson & Reanier, J. Chrom. 255:137-149 (1983).

The DNA sequence encoding for a particular mRNA, a polynucleotide sequence encoding a protein of interest having a known amino acid sequence, including its variants or mutants, and synthetic oligonucleotides can be verified after cloning or subcloning using, e.g., the chain termination method for sequencing double-stranded templates of Wallace et al., Gene 16:21-26 (1981).

### III. Modified Poly(A) Sequences

The present inventors discovered earlier that, upon the poly(A) tail sequence of an mRNA molecule being modified by incorporating a certain number of cytosines (Cs) in place of the adenines (As) near the 3′ end of the tail sequence, the mRNA molecule becomes more stable and can lead to increased protein expression from the coding sequence carried by the mRNA. The earlier disclosure can be found in WO2022/028559. Their later studies further reveal that a modified poly(A) tail sequence fitting a particular profile is capable of very significantly increase the recombinant expression of the protein the mRNA encodes and is suitable for use in the form of a self-amplifying RNA (saRNA) for various therapeutic or prophylactic purposes such as vaccination (see, e.g., WO2024/188312 and WO2025/011636). In this disclosure, the inventors show that further improvement is effectuated by the inclusion of a “linker,” which is a short stretch of nucleotide bases randomly selected from A, C, G, and T/U and placed upstream from the C substitutions in the modified poly(A) tail, in aspects of DNA template replication and recombination protein productions.

Briefly, WO2024/188312 indicates that very significant increase in protein expression, e.g., at least 100% or up to 500% increase, may be achieved, when a modified poly(A) tail is incorporated into an mRNA molecule encoding a protein of interest. Typically, the modified poly(A) tail sequence is in the overall length of about 40 to 150 nucleotides, e.g., about 60 to 120, or about 80 to 100, or about 80, 90, or 100 nucleotides in total length. The first segment of the modified poly(A) tail sequence starting from its 5′ end consists entirely of a string of As, typically ranging from about 30 to 100 nucleotides in length, e.g., about 60 to 100, or about 70 to 90, or about 70, 80, or 90 As in total length. The second segment, immediately to the 3′ end of the first segment, consists entirely of a string of Cs, typically ranging from about 1 to 40 nucleotides in length, e.g., about 5 to 40, about 10 to 35, about 12 or 10 to 30, or about 15 to 25 Cs in total length. In most cases, the second segment is no more than 30% of the total length of the modified poly(A) tail sequence, e.g., no more than ¼ or ⅕ of the length of the first segment. The third and the last segment of the modified poly(A) tail sequence is located at the 5′ end of the sequence and consists of at least one A but no cytosine. For example, this segment may have 1-5 consecutive As without any C substitution.

Further modifications and improvement to the artificial poly(A) tail sequence are described in WO2025/011636: in addition to adenine to cytosine substitutions, adenine residues may be substituted with one or more other nucleotides, such as guanine (G) and thymine (T)/uracil (U). The artificial poly(A) sequence is generally described as having about 30-150 As, with at least 1 A substituted with a C and at least 1 A substituted with 1 G or T/U in the last ⅓ portion of the artificial poly(A) sequence at its 3′ end, for example, having about 30 As at its 5′ end, 1 A at its 3′ end, with a stretch of about 8 nucleotides in between—at least 1 of which is a C and the rest G or T/U. Exemplary artificial poly(A) tail sequences disclosed therein are characterized as 31A8CA, 30AG8CA, 30A4CG4CA, 30A8CGA, 30AU8CA, 30A4CU4CA, and 30A8CUA from its 5′ end to its 3′ end.

In contrast to WO2024/188312 and WO2025/011636, the artificial poly(A) tail sequence of the present invention features not only a substantial stretch of A to C substitutions near the 3′ end (while the last 1-5 nucleotides remain A) but also a middle segment of a so-called linker sequence of about 5-20 randomly selected nucleotides (i.e., which may be independently A, C, G, or T/U), immediately following the opening segment of a plurality of As (e.g., about 20-60 As) at the 5′ end of the artificial poly(A) sequence and immediately followed by another segment of a string of As (e.g., about 30-90 As), which is in turn followed by a string of C (e.g., about 5-40 Cs) plus at least 1 and no more than 5 As (e.g., 1 A) at the 3′ end of the artificial poly(A) sequence.

In some embodiments, the present invention describes an artificial poly(A) sequence that includes, from its 5′ end to its 3′ end, 5 distinct segments: (1) a first segment of a consecutive string of about 20-60 adenines: (2) a second segment (i.e., a linker sequence) of about 5-20 nucleotides, each of which is a randomly and independently selected nucleotide from adenine (A), cytosine (C), guanine (G), and thymine (T)/uracil (U): (3) a third segment of another consecutive string of about 30-90 adenines: (4) a fourth segment of a consecutive string of about 5-40 cytosines; and (5) a fifth segment at the 3′ end of the artificial poly(A) sequence, consisting of 1-5 adenines.

In some embodiments, the total length of the artificial poly(A) sequence of this invention ranges from about 60 to about 200, about 80 to about 150, or about 90 to about 120, e.g., about 100 or 110 nucleotides. On the other hand, the total number of cytosines (i.e., in the 2nd and 4th segments) in this artificial poly(A) sequence is no more than ⅓ of the total number of nucleotides in this artificial poly(A) sequence, for example, the total number of cytosines in the 4th segment of this artificial poly(A) sequence is no more than 30% of the total number of this artificial poly(A) sequence, no more than about 20, 30, 40, 50, or 60 Cs, e.g., about 5, 6, 7, 8, 9, 10, 11, 12, 13, 14, 15, 16, 17, 18, 19, 20, 21, 22, 23, 24, 25, 26, 27, 28, 29, or 30 consecutive Cs in the 4th segment of the artificial poly(A) sequence.

In some embodiments, the artificial poly(A) sequence has a string of about 20-60 or about 25-50 adenines in its 1^{st} segment located at the 5′ end of the artificial poly(A) sequence. For example, there may be about 25 to about 40, about 30 to about 40, or about 30 As in the 1^{st} segment, e.g., about 25, 26, 27, 28, 29, 30, 31, 32, 33, 34, 35, 36, 37, 38, 39, 40, 41, 42, 43, 44, or 45 consecutive As in the segment.

In some embodiments, the 2^{nd} segment of the artificial poly(A) sequence is a so-called linker sequence, which is a string of about 5-20 or about 7-15 random nucleotides, each could be an independently selected nucleotide of A, C, G, or T/U. For example, the linker sequence may be about 8-12 random nucleotides or about 10 random nucleotides, e.g., about 5, 6, 7, 8, 9, 10, 11, 12, 13, 14, 15, 16, 17, 18, 19, or 20 random nucleotides. One exemplary linker sequence is shown as SEQ ID NO:5 in Table 1.

In some embodiments, the 3^{rd} segment of the artificial poly(A) sequence is a string of consecutive adenines of about 30-90, about 40-80, or about 60 in number, for example, about 40, 41, 42, 43, 44, 45, 46, 47, 48, 49, 50, 51, 52, 53, 54, 55, 56, 57, 58, 59, or 60 adenines.

In some embodiments, the 4^{th} segment of the artificial poly(A) sequence is a string of consecutive cytosines of about 5-40, about 7-20, about 8-15, about 9-12, or about 10 in number. For example, about 5, 6, 7, 8, 9, 10, 11, 12, 13, 14, 15, 16, 17, 18, 19, 20, 21, 22, 23, 24, 25, 26, 27, 28, 29, or 30 consecutive cytosines may be present in this segment of the artificial poly(A) sequence.

In some embodiments, the 5^{th} and last segment of the artificial poly(A) sequence consists of about 1-5 or about 1-3 adenines at the 3′ end of the artificial poly(A) sequence. For example, the artificial poly(A) sequence of this invention may have one single adenine at its 3′ end immediately adjacent to the 4^{th} segment of a string of cytosines. In other cases, there may be 1-5 adenines, e.g., 1, 2, 3, 4, or 5 adenines, at the 3′ end of the artificial poly(A) sequence following the 4^{th} segment of a string of cytosines.

In some embodiments, the artificial poly(A) sequence consists of a total 110 nucleotides: 30 As at the 5′ end, followed by a 10-nucleotide linker, a string of 59 As, a string of 10 Cs, and 1 A at the 3′ end. One exemplary artificial poly(A) sequence has the nucleotide sequence set forth in SEQ ID NO:4 in Table 1.

The present invention also provides polynucleotide sequences, both in the form of DNA and RNA, comprising the modified poly(A) tail sequence as described above and herein. These sequences may also include a coding sequence for a protein of interest, which may be a bioactive agent, e.g., a protein of therapeutic function and thus useful for disease treatment (such as gene therapy for cancer or other diseases) or a protein derived from a pathogen and thus useful as an vaccine (such as for immunization against an infectious disease). The coding sequence is located immediately adjacent to the 5′ end of the modified poly(A) tail sequence of this invention.

### IV. Expression Cassettes and Vectors

The disclosure also provides expression cassettes comprising a promoter and an artificial poly(A) sequence described herein. Such an expression cassette, especially in the form of a replicable vector (e.g., a DNA plasmid or a viral vector), is useful tool for the cloning/subcloning and expression of any coding sequence for a protein. Thus, in some cases, the expression cassette can further comprise a polynucleotide sequence encoding one or more polypeptides between the promoter and the artificial poly(A) sequence, wherein the polynucleotide coding sequence is operably linked to the promoter and the artificial poly(A) sequence. In some embodiments, the expression cassette can further comprise a multiple cloning site between the promoter and the artificial poly(A) sequence. Moreover, the expression cassette can further comprise a transcription initiation codon and a transcription termination codon, both of which can be operably linked to the promoter and the artificial poly(A) sequence, as well as any potential coding sequence to be introduced in between the promoter and the modified poly(A) tail sequence by way of using one or more of the multiple cloning sites. Additional elements such as transcriptional activation or enhancer sequences may be included in the expression cassettes and vectors.

In some embodiments, the promoter may be homologous or heterologous to the polynucleotide coding sequence between the promoter and the artificial poly(A) sequence. In some embodiments, the promoter may be inducible. In some embodiments, the promoter may be cell or tissue-specific. In some embodiments, the promoter may be a constitutive promoter. In some embodiments, the expression cassette can be expressed specifically in certain cell and/or tissue types within one or more organs. Alternatively, the expression cassette can be expressed constitutively (e.g., using a constitutive promoter). Further, an expression cassette can contain a marker gene that confers a selectable phenotype on transfected cells. For example, the marker may encode antibiotic resistance, such as resistance to kanamycin, G418, bleomycin, or hygromycin.

The disclosure also provides expression vectors comprising the expression cassette. The expression vectors serve as vehicles that can deliver the expression cassettes into the targeted destination, e.g., inside cells. The expression vectors can be transfected into cells. Techniques for transfecting a wide variety of cells are well known and described in the technical and scientific literature. See, e.g., Kim and Eberwine, Anal Bioanal Chem. 397 (8): 3173-8, 2020. The disclosure also provides a host cell that comprises the expression cassette or the vector described herein. Once transfected into target cells, the polynucleotide encoding one or more polypeptides and the artificial poly(A) sequence can be transcribed into an RNA polynucleotide sequence.

### V. Modifications

An artificial poly(A) sequence of the present invention as described above and herein or a polynucleotide containing such an artificial poly(A) sequence can contain other modifications to improve its stability.

Modifications of mRNA structural elements have been investigated to improve the stability and translational efficiency. These modifications include 5′cap modification, artificial 5′ and 3′ UTR sequences, and a coding region with codon optimization. Further, chemical modifications of mRNA molecules, including the use of pseudouridine and 5-methyl-cytosine, have been observed to increase protein translation while reducing immune response.

### Modified Nucleobases

An artificial poly(A) sequence described herein or an RNA polynucleotide containing an artificial poly(A) sequence described herein can contain one or more modified nucleobases. A modified nucleobase (or base) refers to a nucleobase having at least one change that is structurally distinguishable from a naturally-occurring nucleobase (i.e., adenine, guanine, cytosine, thymine, or uracil). In some embodiments, a modified nucleobase is functionally interchangeable with its naturally-occurring counterpart. Both naturally-occurring and modified nucleobases are capable of hydrogen bonding. Modified nucleobases may help to improve the stability of a polynucleotide, such as increasing its half-life and preventing intracellular degradation and proteolytic cleavage. In some embodiments, an artificial poly(A) sequence described herein or an RNA polynucleotide containing an artificial poly(A) sequence described herein may include at least one modified nucleobase. Examples of modified nucleobases include, but are not limited to, 5-methylcytosine, 5-hydroxymethyl cytosine, xanthine, hypoxanthine, 2-aminoadenine, 6-methyladenine, 6-methylguanine, 2-propyladenine, 2-propylguanine, 2-thiouracil, 2-thiothymine, 2-thiocytosine, 5-halouracil, 5-halocytosine, 5-propynyluracil, 5-propynylcytosine, 6-azouracil, 6-azocytosine, 6-azothymine, 5-uracil (pseudouracil), 4-thiouracil, 8-haloadenine, 8-aminoadenine, 8-thioladenine, 8-thioalkyladenine, 8-hydroxyladenine, 8-haloguanine, 8-aminoguanine, 8-thiolguanine, 8-thioalkylguanine, 8-hydroxylguanine, 5-halouracil, 5-bromouracil, 5-trifluoromethyluracil, 5-halocytosine, 5-bromocytosine, 5-trifluoromethylcytosine, 7-methylguanine, 7-methyladenine, 2-fluoroadenine, 2-aminoadenine, 8-azaguanine, 8-azaadenine, 7-deazaguanine, 7-deazaadenine, 3-deazaguanine, and 3-deazaadenine.

### Modified Sugars

An artificial poly(A) sequence of this invention as described above or herein, or an RNA polynucleotide containing such an artificial poly(A) sequence, can also contain one or more modified sugars. A modified sugar refers to a sugar having at least one change that is structurally distinguishable from a naturally-occurring sugar (i.e., ribose in RNA). Modifications on modified sugars may help to improve the stability of an artificial poly(A) sequence described herein or an RNA polynucleotide containing an artificial poly(A) sequence described herein. In some embodiments, the sugar is a pentofuranosyl sugar. The pentofuranosyl sugar ring of a nucleoside may be modified in various ways including, but not limited to, addition of a substituent group, particularly, at the 2′ position of the ring: bridging two non-geminal ring atoms to form a bicyclic sugar (i.e., a locked sugar); and substitution of an atom or group such as —S—, —N(R)— or —C(R₁) (R₂) for the ring oxygen. Examples of modified sugars include, but are not limited to, substituted sugars, especially 2′-substituted sugars having a 2′-F, 2′-OCH₂ (2′-OMe), or a 2′-O(CH₂) 2-OCH₃ (2′-O-methoxyethyl or 2′-MOE) substituent group; and bicyclic sugars. A bicyclic sugar refers to a modified pentofuranosyl sugar containing two fused rings. For example, a bicyclic sugar may have the 2′ ring carbon of the pentofuranose linked to the 4′ ring carbon by way of one or more carbons (i.e., a methylene) and/or heteroatoms (i.e., sulfur, oxygen, or nitrogen). The second ring in the sugar limits the flexibility of the sugar ring and thus, constrains the oligonucleotide in a conformation that is favorable for base pairing interactions with its target nucleic acids. An example of a bicyclic sugar is a locked sugar, which is a pentofuranosyl sugar having the 2′-oxygen linked to the 4′ ring carbon by way of a carbon (i.e., a methylene) or a heteroatom (i.e., sulfur, oxygen, or nitrogen). In some embodiments, a locked sugar has the 2′-oxygen linked to the 4′ ring carbon by way of a carbon (i.e., a methylene). In other words, a locked sugar has a 4′-(CH₂)—O-2′ bridge, such as α-L-methyleneoxy (4′-CH₂—O-2′) and β-D-methyleneoxy (4′-CH₂—O-2′). A nucleoside having a lock sugar is referred to as a locked nucleoside.

Other examples of bicyclic sugars include, but are not limited to, (6'S)-6′ methyl bicyclic sugar, aminooxy (4′-CH₂—O—N(R)-2′) bicyclic sugar, oxyamino (4′-CH₂—N(R)—O-2′) bicyclic sugar, wherein R is, independently, H, a protecting group or C1-C12 alkyl. The substituent at the 2′ position can also be selected from allyl, amino, azido, thio, O-allyl, O—C1-C10 alkyl, OCF3, O(CH₂)₂SCH₃, O(CH₂)₂—O—N(Rₘ)(Rₙ), and O—CH₂—C(═O)—N(Rₘ)(Rₙ), wherein each Rₘ and Rₙ is, independently, H or substituted or unsubstituted C1-C10 alkyl.

In some embodiments, a modified sugar is an unlocked sugar. An unlocked sugar refers to an acyclic sugar that has a 2′, 3′-seco acyclic structure, where the bond between the 2′ carbon and the 3′ carbon in a pentofuranosyl ring is absent.

### Modified Internucleoside Linkages

An artificial poly(A) sequence of this invention as described above or herein, or an RNA polynucleotide containing such an artificial poly(A) sequence, can also contain one or more internucleoside linkages. An internucleoside linkage refers to the backbone linkage that connects the nucleosides. An internucleoside linkage may be a naturally-occurring internucleoside linkage (i.e., a phosphate linkage, also referred to as a 3′ to 5′ phosphodiester linkage, which is found in DNA and RNA) or a modified internucleoside linkage. A modified internucleoside linkage refers to an internucleoside linkage having at least one change that is structurally distinguishable from a naturally-occurring internucleoside linkage. Modified internucleoside linkages may help to improve the stability of an artificial poly(A) sequence described herein or an RNA polynucleotide containing an artificial poly(A) sequence described herein.

Examples of modified internucleoside linkages include, but are not limited to, a phosphorothioate linkage, a phosphorodithioate linkage, a phosphoramidate linkage, a phosphorodiamidate linkage, a thiophosphoramidate linkage, a thiophosphorodiamidate linkage, a phosphoramidate morpholino linkage, and a thiophosphoramidate morpholino linkage, and a thiophosphorodiamidate morpholino linkage, which are known in the art and described in, e.g., Bennett and Swayze, Annu Rev Pharmacol Toxicol. 50:259-293, 2010. A phosphorothioate linkage is a 3′ to 5′ phosphodiester linkage that has a sulfur atom for a non-bridging oxygen in the phosphate backbone of an oligonucleotide. A phosphorodithioate linkage is a 3′ to 5′ phosphodiester linkage that has two sulfur atoms for non-bridging oxygens in the phosphate backbone of an oligonucleotide. A thiophosphoramidate linkage refers to a 3′ to 5′ phospho-linkage that has a sulfur atom for a non-bridging oxygen and a NH group as the 3′-bridging oxygen in the phosphate backbone of an oligonucleotide. In some embodiments, an artificial poly(A) sequence of this invention as described above and herein, or an RNA polynucleotide containing such an artificial poly(A) sequence has at least one (e.g., at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, or 49 or more) phosphorothioate linkage. In some embodiments, all of the internucleoside linkages in an artificial poly(A) sequence of this invention as described above and herein, or an RNA polynucleotide containing such an artificial poly(A) sequence, are phosphorothioate linkages.

### VI. Methods of Use

The artificial poly(A) sequences of this invention as described above and herein can be used in methods for producing a polypeptide of interest in a cell. The polypeptide of interest, which may be a therapeutic protein as a drug or may be a protein antigen as a vaccine, can be encoded by a polynucleotide sequence that has, at its 3′ end, an artificial poly(A) sequence as described herein. A nucleic acid (e.g., DNA or RNA) comprising the polynucleotide sequence encoding the polypeptide of interest can be delivered into a cell, in which the polypeptide of interest can then be expressed.

The inventors have demonstrated that the artificial poly(A) sequences described herein provide an enhanced performance for the recombinant production of mRNA therapeutics or vaccines with increased protein expression of protein drugs or antigens encoded by the mRNA sequences and thus improved and prolonged treatment efficacy and immune response. Further, despite the enhancement, this technique does not increase the overall cost for mRNA therapeutic/vaccine manufacturing.

In some embodiments, the polypeptide of interest can be an antigen, e.g., a tumor antigen or an antigen from a pathogen such as bacteria, virus, or fungus. The polypeptide of interest can also be a therapeutic protein. The polypeptide of interest encoded by a polynucleotide sequence with an artificial poly(A) sequence of this invention at its 3′ end can be expressed in specific target cell type, for example, an immune cell, such as a dendric cell, a neutrophil, an eosinophil, a basophil, a mast cell, a macrophage, a histiocyte, a B cell, a T cell, a lymphocyte, and a killer cell. In other embodiments, the target cell is a tumor cell. In some embodiments, the polypeptide of interest (e.g., a tumor antigen) can be expressed on the cell surface of the targeted cell type.

The polypeptide of interest encoded by a polynucleotide sequence having an artificial poly(A) sequence of this invention at its 3′ end can be expressed in a cell in the body of a subject. In certain embodiments, the subject has cancer or is at risk of developing cancer. In some embodiments, the subject is exposed to certain infectious pathogens and at risk of becoming infected. The polypeptide of interest as a protein antigen can induce an immune response in vitro or in vivo, namely an immune response against an antigen (e.g., an antigen from a viral, bacterial, or fungal pathogen).

The nucleic acid (e.g., DNA or RNA) comprising the polynucleotide sequence (i.e., comprising at its 3′ end an artificial poly(A) sequence of this invention as described above and herein) encoding the polypeptide of interest can further comprise a promoter. Further, the presence of a multiple cloning site between the promoter and the artificial poly(A) sequence allows future cloning/engineering by way of inserting one or more polynucleotide sequences encoding any additional polypeptide(s) of interest, thus supporting a versatile use of this expression system taking advantage of this discovery.

### VII. Pharmaceutical Compositions and Administration

The disclosure provides pharmaceutical compositions comprising a nucleic acid (e.g., DNA or RNA) comprising the polynucleotide sequence encoding the polypeptide(s) of interest, or an expression cassette or vector comprising thereof, in which the polynucleotide sequence comprises an artificial poly(A) sequence described herein at its 3′ end. Suitable formulations for use in the present invention are found, e.g., in Remington's Pharmaceutical Sciences, Mack Publishing Company, Philadelphia, PA, 17^{th} ed. (1985). For a brief review of methods for drug delivery, see, Langer, Science 249:1527-1533 (1990). The pharmaceutical compositions can be administered by various routes, e.g., systemic administration via oral ingestion or injection (e.g., intravenous, intramuscular, or subcutaneous injection) as well as local delivery such as by intratumoral, intracranial, or intraperitoneal injection or by direct (e.g., topical) application or by using an appropriate suppository. One preferred route of administering the pharmaceutical compositions is intravenous administration. In some embodiments, intravenous administration is performed at daily doses of about 1 to about 1000 μg, about 5 to about 500 μg, about 10 to about 250 μg, about 20 to about 100 μg, or about 25 to about 50 μg of the RNA of this invention. Additionally, the composition may be formulated in a daily, weekly, or monthly dosage format for administration to the subject. The appropriate dose may be administered in a single, one-time daily dose or as divided doses presented at appropriate intervals, for example one dose every two, three, four, five, six, or more months such as every 12 months. Single or multiple administrations of the compositions can be carried out with dose levels and pattern being selected by the treating physician.

For preparing pharmaceutical compositions, one or more inert and pharmaceutically acceptable carriers are used. Depending on the means of administration, the pharmaceutical carrier can be either solid or liquid. Solid form preparations include, for example, powders, creams/pastes, tablets, dispersible granules, capsules, cachets, and suppositories. A solid carrier can be one or more substances that can also act as diluents, flavoring agents, solubilizers, lubricants, suspending agents, binders, or tablet disintegrating agents: it can also be an encapsulating material. Powders and other versions of solid compositions contain an adequate amount of the active ingredient(s) (e.g., the mRNA of the present invention) along with one or more carriers. Suitable carriers include, for example, magnesium carbonate, magnesium stearate, talc, lactose, sugar, pectin, dextrin, starch, tragacanth, methyl cellulose, sodium carboxymethyl cellulose, a low-melting wax, cocoa butter, and the like.

Liquid pharmaceutical compositions include, for example, solutions suitable for oral or intranasal administration or local delivery, suspensions, and emulsions suitable for oral administration. Sterile water solutions of the active component (e.g., the polypeptide of interest, especially one with therapeutic activity) or sterile solutions of the active component in solvents comprising water, buffered water, saline, PBS, ethanol, or propylene glycol are examples of liquid or semi-liquid compositions suitable for oral administration or local delivery such as by topical application or rectal suppository. The compositions may contain pharmaceutically acceptable auxiliary substances as required to approximate physiological conditions, such as pH adjusting and buffering agents, tonicity adjusting agents, wetting agents, detergents, and the like.

Sterile solutions can be prepared by dissolving the active component in the desired solvent system, and then passing the resulting solution through a membrane filter to sterilize it or, alternatively, by dissolving the sterile active component in a previously sterilized solvent under sterile conditions. The resulting aqueous solutions may be packaged for use as is, or lyophilized, the lyophilized preparation being combined with a sterile aqueous carrier prior to administration. The pH of the preparations is typically between about 3 and about 11, for example, from about 5 to about 9, or from about 7 to about 8.

In some embodiments, the composition can be formulated as a composition of nucleic acid particles, especially in the form of lipid nanoparticles (LNP) comprising the RNA. One or more types of lipid, as well as other ingredients, may be used in the formulation. For example, the LNP may comprise a cationic lipid, a neutral lipid, a steroid, a polymer conjugated lipid, and the RNA. In some cases, the LNP may further comprise at least one lipid or lipid-like material other than a cationic or cationically ionizable lipid or lipid-like material, at least one polymer other than a cationic polymer, or a mixture thereof. In some embodiments, the ratio of mRNA to total lipid (N/P) is between 5 and 10 such as about 6 or about 7. Nucleic acid particles of this invention may have an average diameter ranging from about 30 nm to about 1000 nm, from about 50 nm to about 800 nm, from about 70 nm to about 600 nm, from about 90 nm to about 400 nm, or from about 100 nm to about 300 nm. The nucleic acid particles may exhibit a polydispersity index less than about 0.5, less than about 0.4, less than about 0.3, or about 0.2 or less. By way of example, the nucleic acid particles can exhibit a polydispersity index in a range of about 0.1 to about 0.3 or about 0.2 to about 0.3.

In certain embodiments, a nucleic acid (e.g., DNA or RNA) construct of this invention as described above and herein comprising a polynucleotide sequence having an artificial poly(A) sequence of this invention at its 3′ end and encoding a protein of interest (e.g., an antigen derived from an infectious pathogen) is used as a vaccine, for example, to elicit in recipients a desired immune response against the protein antigen, thus potentially provide protection against future infection by the pathogen. For compositions comprising the nucleic acid construct of this invention intended for use for vaccination purposes, they often further comprise one or more adjuvants (e.g., starch, pregelled starch, calcium phosphate mannitol, lactose, saccharose, glucose, sorbitol, microcrystalline cellulose, gelatin, polyvinylpyrrolidone, methylcellulose, ethylcellulose, arabic gum, tragacanth gum, magnesium stearate, stearic acid, colloidal silica, glyceryl monostearate, hydrogenated castor oil, waxes, and mono-, bi-, and trisubstituted glycerides). A vaccine or a composition containing the nucleic acid construct of this invention can be formulated in accordance with the intended delivery method, e.g., for injection such as intramuscular or subcutaneous injection, or for mucosal delivery such as by oral ingestion, nasal inhalation, or as eye drop, etc.

### Examples

The following examples are provided by way of illustration only and not by way of limitation. Those of skill in the art will readily recognize a variety of non-critical parameters that could be changed or modified to yield essentially the same or similar results.

### Introduction

The COVID-19 vaccine has brought to light synthetic mRNA as a promising therapeutic modality. It can produce any kind of proteins on demand, be easily manufactured, and minimize the risk of accumulation in cells. Despite all these advantages, current mRNA-based drugs still have some limitations, including 1) potential mutants during scale-up and 2) low protein production efficiency.

Industrial production of therapeutic mRNAs starts from the plasmid, and recombination of the poly(A) tail can happen during bacteria amplification of the plasmid (Trepotec et al., RNA, 25 (4), 507-518, 2019), which destabilizes the plasmid and affects production. In this study and the current design by BNT (Vogel et al., Nature, 592 (7853), 283-289, 2021), a linker is used to reduce the combination rate. However, this only reduces recombination from the plasmid without much improvement in protein production efficiency.

Our previous study and patent have designed an optimized cytidine-containing tail to enhance the protein production of synthetic mRNA. Therefore, we show by combining both the linker and our optimized tail, we are able to not only minimize the recombination of mRNA to stabilize the final product but also enhance the performance of mRNA-based drugs.

### Method

### Transformation

Table 1 shows the nucleotide sequences of poly(A) tails tested in all the samples. All the EGFP-tail constructs were cloned into pUC-GW-Amp (Genewiz, China). Plasmids were transformed into competent E. coli (DH5a) using heat shock method following the manufacturer's protocol (Qiagen, Germany). Transformed E. coli were plated onto LB-ampicillin agar plates. Positive colonies were picked the next day into 5 mL LB medium supplemented with ampicillin and let grow for 24 hours.

| Source of tails | Tail names | Sequences |
|---|---|---|
| Commonly used | 100A | AAAAAAAAAAAAAAAAAAAAAAAAAAAAAAAAAAAAAA |
| on synthetic | | AAAAAAAAAAAAAAAAAAAAAAAAAAAAAAAAAAAAAA |
| mRNA | | AAAAAAAAAAAAAAAAAAAAAAAA |
| | | (SEQ ID NO: 1) |
| BNT (linker | 30AL70A | AAAAAAAAAAAAAAAAAAAAAAAAAAAAAAGCATATGA |
| sequence | | CTAAAAAAAAAAAAAAAAAAAAAAAAAAAAAAAAAAAA |
| SEQ ID NO: 5 | | AAAAAAAAAAAAAAAAAAAAAAAAAAAAAAAAAA |
| underlined) | | |
| | | (SEQ ID NO: 2) |
| Cytidine- | 79A20CA | AAAAAAAAAAAAAAAAAAAAAAAAAAAAAAAAAAAAAA |
| containing | | AAAAAAAAAAAAAAAAAAAAAAAAAAAAAAAAAAAAAA |
| tail | | AAACCCCCCCCCCCCCCCCCCCCA |
| | | (SEQ ID NO: 3) |
| Further- | 30AL59A10CA | AAAAAAAAAAAAAAAAAAAAAAAAAAAAAAGCATATGA |
| engineered | | CTAAAAAAAAAAAAAAAAAAAAAAAAAAAAAAAAAAAA |
| tail (linker | | AAAAAAAAAAAAAAAAAAAAAAACCCCCCCCCCA |
| sequence | | (SEQ ID NO: 4) |
| SEQ ID NO: 5 | | |
| underlined) | | |

Table 1. Poly(A) tail sequences: the sequence for BNT is used from Vogel et al., 2021, supra. The sequence for Cytidine-containing tail is used from Li et al., Molecular Therapy-Nucleic Acids, 30, 300-310, 2022. Current approach adopts the same linker sequence as Vogel et al., 2021, supra.

### Fragmented Tail Analysis

Plasmids from overnight cultured bacteria were purified using QIAprep Spin Miniprep Kit (Qiagen, Germany). The poly(A) tail region was digested by restriction enzymes as described in Trepotec et al., 2019, supra. Digested tails were prepared using DNF-474 HS NGS Fragment Kit (1-6000 bp) (Agilent Technologies, CA, USA), and resolved on Fragment Analyzer (Agilent Technologies, CA, USA). A clear 100-bp band indicates no recombination has occurred, and vice versa.

dsDNA Template Generation and RNA Synthesis

The dsDNA templates were generated by fusion PCR using Q5® High-Fidelity 2× Master Mix (NEB, MA, USA). The PCR products were purified using the QIAquick PCR Purification Kit (Qiagen, Germany). The quality of synthesized templates was assessed via agarose gel electrophoresis and purified using QIAquick Gel Extraction Kit (Qiagen, Germany). The concentration of the purified templates was determined by the Nano Vue Plus spectrophotometer (GE Healthcare, UK). The mRNAs were transcribed from the dsDNA templates using MegaScript T7 Transcription Kit (Thermo Fisher Scientific, MA, USA). The reaction mixtures were purified using the RNeasy MiniElute Cleanup Kit (Qiagen, Germany). The concentration of the product mRNAs was measured by the Nano Vue Plus spectrophotometer (GE Healthcare, UK). The quality of the mRNAs was determined by Urea-PAGE gel electrophoresis.

### Cell Culture and Transfection

HEK293 cells were cultured in Dulbecco's Modified Eagle Medium (DMEM) supplemented with 10% Fetal Bovine Serum (FBS) and 1% Non-Essential Amino Acids (NEAA) at 37° C. with 5% CO₂. Cells were seeded into a 48-well plate at 1× 10⁵ cells/mL one day prior to transfection. Cells were transfected with mRNAs of interest at 100 ng/well using Lipofectamine MessengerMAX Reagent (Thermo Fisher Scientific, MA, USA). For flow cytometry analysis, iRFP mRNA was co-transfected with EGFP mRNA at 15 ng/well for internal reference.

### Flow Cytometry Analysis

All cell samples were analyzed by Attune NxT Flow Cytometer (Thermo Fisher Scientific, MA, USA). The flow cytometry was calibrated with Attune Performance Tracking Beads following the manufacturer's protocol (Thermo Fisher Scientific, MA, USA). After 24 hours of transfection, cells were suspended using 0.25% trypsin, diluted in complete DMEM, and passed through a 35-micron nylon mesh. EGFP/Alexa Fluor 488 signals were detected by excitation laser at 488 nm and emission filter at 530/30 nm. iRFP signals were detected by excitation laser at 637 nm and emission filter at 670/14 nm. The iRFP intensities were used to gate for the positively transfected cell population. The relative EGFP expression was determined through comparison to cells trasfected with EGFP-100A. All the data were analyzed using one-way ANOVA.

### Results

Cytidine-Containing Tails can Reduce Recombination from Bacteria Amplification

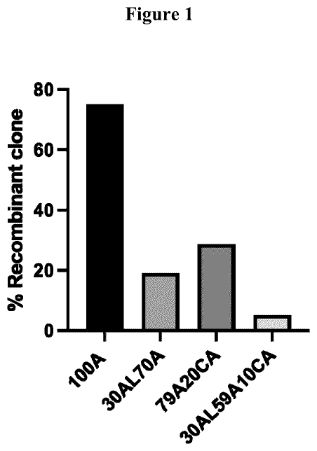

First, we tested the recombination rate of plasmid on the cytidine-containing tails (79A20CA tail, SEQ ID NO:3). This tail is previously proven to be able to significantly enhance mRNA performance. From FIG. 1, this tail alone without any further modification can already reduce the recombination rate to lower than 30% as compare to adenosine only tail (100A, SEQ ID NO:1) having a recombination rate of 75% after 24 hours of amplification in bacteria.

Cytidine-Containing Tail with Linker can Further Reduce Recombination

Next, we evaluated the recombination rate of plasmids carrying the further-engineered and more segmented tail (30AL59A10CA tail, SEQ ID NO:4). From FIG. 1, this tail can further reduce the recombination rate down to 5%. In contrast, the recombination rate of the BNT tail (30AL70A tail, SEQ ID NO:2), which is commonly used in many constructs to minimize recombination, is approximately 20%. This significant reduction indicates that our further-engineered tail can greatly enhance the purity of mRNA produced from plasmids, thereby facilitating downstream applications.

### Modified Tails do not Affect Bacteria Growth

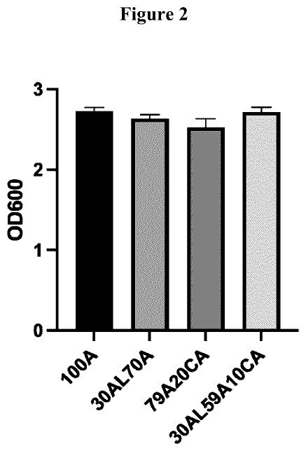

For practical application, we also examined the effect of these plasmids on bacteria growth which subsequently affects the downstream mRNA production. As shown in FIG. 2, all the transformed bacteria showed little-to-no difference in OD600. This indicates that the plasmid has no effect on the growth of the bacteria, and hence, would not affect the yield of plasmid production.

Cytidine-Containing Tail with Linker can Effectively Enhance mRNA Expression

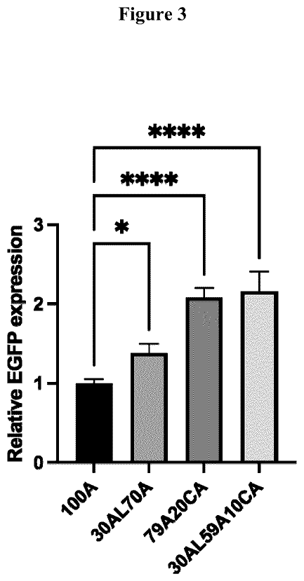

In previous studies, the cytidine-containing tail can effectively enhance mRNA expression. Therefore, we tested the expression level of the new mRNA tail (30AL59A10CA tail, SEQ ID NO:4). As shown in FIG. 3, the further-engineered approach can also enhance the mRNA expression as high as the cytidine-containing tail of the same length (79A20CA, SEQ ID NO:2). As a comparison, the BNT tail (30AL70A tail, SEQ ID NO:2) only enhances the mRNA expression 1.4 times compared to the adenosine-only tail (100A tail, SEQ ID NO:1). Therefore, our new proposed tail can not only minimize the recombination rate in plasmid better than the BNT tail, but also, as an additional benefit, enhance mRNA expression similar to the cytidine-containing tail.

All patents, patent applications, and other publications, including GenBank Accession Numbers and equivalents, cited in this application are incorporated by reference in the entirety for all purposes.

1. An artificial poly(A) sequence having about 20-60 adenines at its 5′ end, about 5-20 random nucleotides, about 30-90 adenines, about 5-40 cytosines, and 1-5 adenines at its 3′ end.

2. The artificial poly(A) sequence of claim 1, wherein the number of cytosines is no more than ⅓ of the total number of nucleotides of the artificial poly(A) sequence.

3. The artificial poly(A) sequence of claim 1, having about 25-50 adenines at its 5′ end, about 7-15 random nucleotides, about 40-80 adenines, about 7-20 cytosines, and 1-3 adenines at its 3′ end.

4. The artificial poly(A) sequence of claim 1, having about 30 adenines at its 5′ end, about 10 random nucleotides, about 60 adenines, and about 10 cytosines in between 1 adenine at its 3′ end.

5. The artificial poly(A) sequence of claim 1, having 30 adenines, 10 random nucleotides, 59 adenines, 10 cytosines, and 1 adenine from its 5′ end to its 3′ end.

6. The artificial poly(A) sequence of claim 1, having the nucleotide sequence set forth in SEQ ID NO:4.

7. The artificial poly(A) sequence of claim 1, which is a DNA sequence.

8. The artificial poly(A) sequence of claim 1, which is an RNA sequence.

9. An expression cassette comprising a promoter and a polynucleotide sequence encoding the artificial poly(A) sequence of claim 1.

10. The expression cassette of claim 9, further comprising a multiple cloning site between the promoter and the polynucleotide sequence encoding the artificial poly(A) sequence.

11. The expression cassette of claim 9, further comprising a transcription initiation codon and a transcription termination codon, both operably linked to the promoter and the polynucleotide sequence encoding the artificial poly(A) sequence.

12. The expression cassette of claim 9, further comprising a polynucleotide sequence encoding one or more polypeptides between the promoter and the artificial poly(A) sequence, wherein the polynucleotide sequence is operably linked to the promoter and the polynucleotide sequence encoding the artificial poly(A) sequence.

13. The expression cassette of claim 9, wherein the artificial poly(A) sequence is set forth in SEQ ID NO:4.

14. A vector comprising the expression cassette of claim 9.

15. A host cell comprising the expression cassette of claim 9.

16. A composition comprising the expression cassette of claim 9.

17. An RNA transcribed from the expression cassette of claim 9.

18. An RNA comprising a coding sequence for one or more polypeptides and the artificial poly(A) sequence of claim 1.

19. The RNA of claim 17, wherein the artificial poly(A) sequence is set forth in SEQ ID NO:4.

20. A composition comprising the RNA of claim 17.

21. The composition of claim 20, further comprising an adjuvant.

22. A method for RNA transcription in a cell or a cell lysate, comprising (i) transfecting the cell with the expression cassette of claim 9; and (ii) cultivating the cell or maintaining the lysate under conditions permissible for RNA transcription from the expression cassette.

23. The method of claim 22, further comprising isolating the RNA transcribed in step (ii).

24. The method of claim 22, wherein the cell is a bacterial cell or the cell lysate is a bacterial cell lysate.

25. A method for recombinant protein expression in a cell, comprising (i) transfecting the cell with the expression cassette of claim 9; and (ii) cultivating or maintaining the cell under conditions permissible for protein expression from the expression cassette.

26. The method of claim 22, wherein the artificial poly(A) sequence is set forth in SEQ ID NO:4.

27. The method of claim 22, wherein the cell is within a human body.


SEQUENCE LISTING (5 sequences)

SEQ ID NO: 1 - RNA, 100 nt, synthetic construct
  misc_feature 1..100: note = Description of sequence: Source of tail: Commonly used on synthetic mRNA; Tail name: 100A
  source 1..100: mol_type = other RNA
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 2 - RNA, 110 nt, synthetic construct
  misc_feature 1..110: note = Description of sequence: Source of tail: BNT; Tail name: 30AL70A
  source 1..110: mol_type = other RNA
aaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 3 - RNA, 100 nt, synthetic construct
  misc_feature 1..100: note = Description of sequence: Source of tail: Cytidine-containing tail; Tail name: 79A20CA
  source 1..100: mol_type = other RNA
aaaaaaaaaaaaaaaaaaaaaaaaaaaaaaaaaaaaaaaaaaaaaaaaaaaaaaaaaaaaaaaaaaaaaaaaaaaaaaacccccccccccccccccccca

SEQ ID NO: 4 - RNA, 110 nt, synthetic construct
  misc_feature 1..110: note = Description of sequence: Source of tail: Further-engineered tail; Tail name: 30AL59A10CA
  source 1..110: mol_type = other RNA
aaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaacccccccccca

SEQ ID NO: 5 - RNA, 10 nt, synthetic construct
  misc_feature 1..10: note = Description of sequence: Linker
  source 1..10: mol_type = other RNA
gcatatgact